(19) United States
(12) Patent Application Publication
(10) Pub. No.: US 2024/0416022 A1
(43) Pub. Date: Dec. 19, 2024

(54) ENDOLUMINAL TREATMENT DEVICES

(71) Applicant: Boston Scientific Scimed, Inc., Maple Grove, MN (US)

(72) Inventors: Rahul PRABHU, Cambridge, MA (US); Paul SMITH, Smithfield, RI (US); Peter L. DAYTON, Brookline, MA (US); Martyn G. FOLAN, Co. Galway (IE); Emma HORGAN, Galway (IE); Hannah ANDERSEN, Brighton, MA (US); Jonathan ROOT, Townsend, MA (US)

(73) Assignee: Boston Scientific Scimed, Inc., Maple Grove, MN (US)

(21) Appl. No.: 18/741,950

(22) Filed: Jun. 13, 2024

Related U.S. Application Data
(60) Provisional application No. 63/508,025, filed on Jun. 14, 2023.

(51) Int. Cl. A61M 1/00; A61M 25/00

(52) U.S. Cl. CPC A61M 1/87; A61M 25/007; A61M 2205/0205

(57) ABSTRACT

A medical device including a distal body coupled to a distal end of a vacuum tube. The distal body may be in fluid communication with a lumen of the vacuum tube. The distal body may include a core having a plurality of openings and one or more layers surrounding the core.

### CROSS-REFERENCE TO RELATED APPLICATIONS

This application claims the benefit of priority to U.S. Provisional Application No. 63/508,025, filed on Jun. 14, 2023, which is incorporated by reference herein in its entirety.

### TECHNICAL FIELD

Various aspects of this disclosure relate generally to minimally invasive medical devices. In particular, aspects of the disclosure relate to medical devices for endoscopic medical procedures, such as closing a wound or otherwise treating tissue.

### BACKGROUND

Endoscopic and open surgical procedures of the gastrointestinal (GI) tract include, for example, colonic resection, bariatric surgery, esophagectomy, gastric bypass, and sleeve gastrectomy, among others. These procedures may result in perforation, post-surgical leaks, or other wounds of the GI tract. Patients with perforations, post-surgical leaks, and/or other wounds in the GI tract have high mortality rates with limited treatment options. Options include endoscopic placement of clips or stents, endoscopic sutures or sealants, or surgical re-operation. Surgery is relatively invasive and has high morbidity and mortality rates. While endoscopic stent placement is a less invasive option, the stent can migrate from the intended location and/or wall off infection at a treatment site, inhibiting drainage.

The medical devices of the current disclosure may rectify some of the deficiencies described above or address other aspects of the art.

### SUMMARY

Each of the aspects disclosed herein may include one or more of the features described in connection with any of the other disclosed aspects.

In an aspect, a medical device may include a distal body coupled to a distal end of a vacuum tube. The distal body may be in fluid communication with a lumen of the vacuum tube. The distal body may include a core including a plurality of openings and one or more layers surrounding the core.

Any of the aspects disclosed herein may include any of the following features, alone or in any combination. At least one of the one or more layers may define a plurality of openings. The plurality of openings of the one or more layers may have a smaller average size than the plurality of openings of the core. The one or more layers may completely surround the core. The one or more layers may surround only a portion of the core. The one or more layers may be coated with an antimicrobial material. At least one of the one or more layers may be coupled to the distal end of the vacuum tube via a clip, drawstring, or suture. At least one of the one more layers may include one or more protrusions extending radially outward from an outer surface of the at least one of the one or more layers. The one or more layers may include a first layer surrounding the core and a second layer surrounding the first layer. An absorbency of the second layer may be greater than an absorbency of the first layer and the absorbency of the first layer may be greater than an absorbency of the core. The core may include a hydrophobic material, and the first layer and the second layer may include a hydrophilic material. A proximal portion of an outer surface of the second layer may include an adhesive. The one or more layers may be coated with a material comprising alginate. The core may include a first channel and a second channel. The first channel and the second channel may extend from a proximal end of the core to a distal end of the core. A distal end of each of the first channel and the second channel may terminate proximal of a distalmost end of the core. The first channel and the second channel may converge at the proximal end of the core to form a third channel.

In another example, a medical device may include a distal body coupled to a distal end of a vacuum tube. The distal body may be in fluid communication with a lumen of the vacuum tube. The distal body may include a core and at least one layer surrounding the core. The core may include a plurality of openings disposed on an outer surface of the core, a first channel, and a second channel. The first channel and the second channel may converge at a proximal end of the core to form a third channel.

Any of the aspects disclosed herein may include any of the following features, alone or in any combination. A distal end of each of the first channel and the second channel may be closed. The third channel may be adjacent to the lumen of the vacuum tube.

In a further aspect, a medical device may include a distal body coupled to a distal end of a vacuum tube. The distal body may include a core having a plurality of openings in fluid communication with a lumen of the vacuum tube. The distal body may further include a first layer surrounding the core and a second layer surrounding the first layer. Each of the first layer and the second layer may include a plurality of openings. The plurality of openings of each of the first layer and the second layer may have a smaller average size than the plurality of openings of the core. An outer surface of the second layer may include a plurality of protrusions extending radially outward.

### BRIEF DESCRIPTION OF THE DRAWINGS

The accompanying drawings, which are incorporated in and constitute a part of this specification, illustrate various aspects of this disclosure and together with the description, serve to explain the principles of the disclosure.

FIGS. 1A and 1B illustrate an exemplary medical device, according to aspects of this disclosure.

FIG. 2 illustrates another exemplary medical device, according to aspects of this disclosure.

FIG. 3 illustrates a further exemplary medical device, according to aspects of this disclosure.

### DETAILED DESCRIPTION

Particular aspects of the present disclosure are described in greater detail below. The terms and definitions provided herein control, if in conflict with terms and/or definitions incorporated by reference.

The terms “proximal” and “distal” are used herein to refer to the relative positions of the components of exemplary medical devices. As used herein, “proximal” refers to a position relatively closer to the exterior of the body or closer to an operator using the medical device. In contrast, “distal” refers to a position relatively further away from the operator using the medical device, or closer to the interior of the body.

As used herein, the terms “comprises,” “comprising,” “including,” “includes,” having,” “has” or any other variation thereof, are intended to cover a non-exclusive inclusion, such that a process, method, article, or apparatus that comprises a list of elements does not include only those elements, but may include other elements not expressly listed or inherent to such process, method, article, or apparatus. The term “exemplary” is used in the sense of “example,” rather than “ideal.”

Further, relative terms such as, for example, “about,” “substantially,” “approximately,” etc., are used to indicate a possible variation of +10% in a stated numeric value or range.

Endoluminal vacuum therapy (EVT or EVAC, and referred to herein as EVAC) is a procedure to treat wounds, such as post-surgical leaks or perforations in the gastrointestinal tract (GI) following a surgical or endoscopic procedure, such as colonic resection, bariatric surgery, or esophagectomy. In EVAC, negative pressure is delivered to the wound site in the GI tract, for example, through a nasogastric tube having a sponge/foam (e.g., vacuum sealed foam) at its distal end. A proximal end of the tube may be connected to a collection container in conjunction with the negative pressure source. The foam is placed endoscopically into the perforation, leak, or other wound. In some examples, EVAC includes endoluminal placement of a foam or other like material into the wound (e.g., target) site, including a perforation, a leak, a cyst, an anastomosis, etc. Placement of the material may be via a catheter, scope (endoscope, bronchoscope, colonoscope, duodenoscope, gastroscope, etc.), tube, or sheath, inserted into the GI tract via a natural orifice. The orifice can be, for example, the nose, mouth, or anus, and the placement can be in any portion of the GI tract, including the esophagus, stomach, duodenum, large intestine, or small intestine. Placement of the material can also be in other organs reachable via the GI tract (e.g., colon, biliary tract, appendix). Negative pressure then is applied.

The foam in the wound, along with the negative pressure, may accelerate healing by encouraging local tissue granulation at a wound site. The foam may be replaced with increasingly smaller volumes of foam as the wound heals and closes. Devices and systems suited for EVAC are limited. For example, EVAC typically requires a foam to be replaced every 3 to 5 days to reduce the risk of tissue ingrowth. Furthermore, infection of the wound may occur, which may prolong treatment of the wound.

Aspects of this disclosure include devices to reduce the number of foam exchanges during EVAC. Components of the devices described herein may be packaged as a kit for EVAC. For example, the devices disclosed herein may include a distal body coupled to a distal end of a tube. The distal body may include a plurality of materials. For example, the distal body may include multiple layers of material. The distal body may include a central sponge/foam surrounded by one or more other materials that cover at least a portion of the sponge. In some aspects, the distal body may include a textured outer layer (e.g., drape) that may inhibit tissue ingrowth through the distal body. In additional or alternative aspects, the distal body may include, for example, layers with antibacterial or antimicrobial features, collagen to promote tissue growth, enhanced absorbency, features to provide storage or collection of exudates, features to assist with removal of the distal body, anti-migration features, and/or hydrophilic or hydrophobic properties. Aspects of the distal body may provide for improved absorption, promotion of fluidic flow, promotion of tissue granulation, prevention or inhibition of tissue in-growth, prevention or inhibition of infection, and/or assistance in drying,

Reference will now be made in detail to examples of the present disclosure described above and illustrated in the accompanying drawings. Wherever possible, the same reference numbers will be used throughout the drawings to refer to the same or like parts.

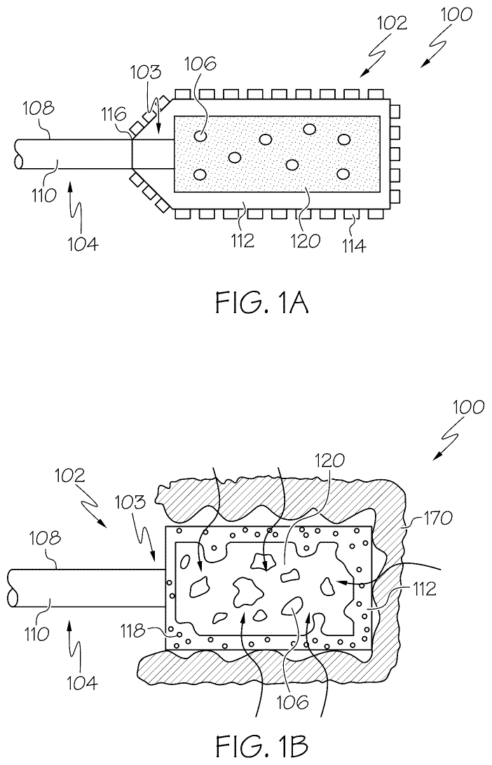

FIGS. 1A and 1B illustrate a distal end of an exemplary EVAC device 100 that may be inserted into a patient for treatment of wounds or otherwise used to help treat tissue. FIGS. 1A and 1B show a cross-sectional view of EVAC device 100. EVAC device 100 may include a distal body 102 and a vacuum tube 104 or other type of conduit. Distal body 102 may be fixedly or removably coupled to a distal end 103 of vacuum tube 104.

Distal body 102 may include a core 120, which may include, for example, a foam, such as a sponge. Core 120 may have any features of any foam that is known in the art for use in EVAC procedures. For example, core 120 may include an open-cell foam. Core 120 may include openings 106 on an outer surface and/or interior thereof. Openings 106 may be any hole, pore, or channel. Openings 106 may include interconnecting channels and/or pores throughout core 120. For example, openings 106 may be pores of a foam of core 120. Openings 106 may have different sizes and shapes. Features of openings 106 (e.g., a size and shape of pores of a foam of core 120) may be selected based on a location of treatment within the body, properties of a wound to be treated, a stage of treatment, or other factors. Distal body 102, including core 120, is illustrated as having a cuboidal shape, but may be any shape, including spherical, cylindrical, irregular, or the like. In some examples, the size of openings 106 may be approximately 500 μm to approximately 1.0 mm in diameter. Openings 106 may be of uniform or non-uniform size and, in some examples, may be larger than 1.0 mm. Distal body 102 may be compressed into a lower profile during insertion to a target site and may expand during deployment at a target site.

Core 120 may include any suitable biocompatible material that may absorb liquids and/or permit liquid or other materials to pass therethrough via, e.g., negative pressure applied to distal body 102. The material of core 120 may be flexible, compressible, porous, hydrophilic, sterile, and/or disposable. For example, a material of core 120 may be an open-cell foam. Suitable materials include polyurethanes, esters, ethers, composite materials, and/or other medical-grade materials.

Vacuum tube 104 may include an outer wall 108 defining one or more lumens 110. Lumen 110 may be open at both a proximal end (not shown) and distal end 103 of vacuum tube 104 within or otherwise coupled to distal body 102. In some examples, outer wall 108 may include a plurality of holes around a circumference of the distal end 103 of vacuum tube 104 and in fluid communication with lumen 110, which may increase the flow of fluid or material into lumen 110. Distal end 103 of vacuum tube 104 may be attached to distal body 102 via sutures or other ties, an adhesive, a shrink-wrapped material, elastic(s), or the like. In one example, a recess (not shown) may be provided in core 120 (e.g., a proximal end of core 120) and/or another portion of distal body 102 to receive distal end 103 of vacuum tube 104. The proximal end of vacuum tube 104 may be connected to a vacuum source (not shown), which may supply a negative pressure to distal body 102. For example, a negative pressure of approximately 125 mm Hg, or approximately 2.5 pounds per square inch (PSI), may be supplied to distal body 102, including core 120. Other suitable amounts of negative pressure may be used. The negative pressure may pull fluid, material, and/or other debris into lumen 110 of vacuum tube 104 via openings 106 of core 120, which may promote healing of a target site.

Distal body 102 may further include a cover 112 surrounding or covering an entirety or a portion of core 120 and, optionally, a portion of distal end 103 of vacuum tube 104. Cover 112 may include perforations 118 therein, such as micro-perforations. Perforations 118 may extend through an entire thickness of cover 112 (or may be in fluid communication with one another so as to provide a path through a wall of cover 112). Perforations 118 of cover 112 may be sized so as to be large enough to allow for fluid flow through perforations 118, while being small enough so as to prevent or inhibit tissue ingrowth into cover 112. Similar to openings 106, the size of perforations 118 may be approximately 500 μm to approximately 1.0 mm in diameter. In some examples, the size of openings 106 may be larger than the size of perforations 118. Cover 112 may have properties such that absorbent properties of core 120 are preserved, with cover 112 forming a protective layer around core 120. A material of cover 112 may include polyurethane ether, polyurethane ester, polyvinyl alcohol, expanded polytetrafluoroethylene (PTFE), perforated PTFE, polyethylene, ChronoFlex C®, and/or polyvinylidene fluoride (PVDF).

A proximal end of cover 112 may include an opening 116, such that a distal end of core 120 may be inserted into opening 116 of cover 112. Cover 112 may then be pulled proximally over distal body 102 (or distal body 102 may be moved distally into cover 112) to cover core 120 (e.g., completely or partially cover core 120) and, optionally, a portion of distal end 103 of vacuum tube 104. Cover 112 may then be secured to distal end 103 of the vacuum tube 104 or core 120 via a clip, drawstring, elastic, suture(s), heat shrinkable capture, or the like. In some examples, cover 112 may be provided as part of an EVAC kit packaged with core 120 and vacuum tube 104. A user may install cover 112 as described above. In other examples, EVAC device 100 may come with cover 112 already in position. In some examples, cover 112 may be fixedly coupled to core 120. In other examples, cover 112 may be removably coupled to core 120.

In some examples, cover 112 may optionally include protrusions 114 extending radially outward from an outer surface of distal body 102 (or from an outer surface of cover 112). Protrusions 114 may help promote healing of a wound by inducing tissue inflammation and tissue granulation as protrusions 114 contact and/or brush up against a tissue wall of a target site. Protrusions 114 may be made from a same material as a remainder of cover 112. Alternatively, protrusions 114 may be made from a different material. Protrusions 114 may have any suitable shape and size. Although protrusions 114 are not shown in FIG. 1B, it will be appreciated that protrusions 114 may be disposed on cover 112 of FIG. 1B. Alternatively, protrusions 114 may be omitted.

FIG. 1B illustrates distal body 102 (including core 120 and cover 112) positioned at a target site 170. When negative pressure is supplied to vacuum tube 104, the negative pressure may pull fluid or material into lumen 110 via openings/perforations 118 of cover 112 and openings 106 of core 120. The arrows shown in FIG. 1B depict a flow of fluid or material through openings/perforations 118 and openings 106. Cover 112 may provide, e.g., a protective layer around core 120 by, e.g., preventing tissue growth into distal body 102 without, e.g., inhibiting fluid flow from target site 170 into distal body 102. As shown in FIG. 1B, tissue may not grow into cover 112 or core 120.

FIG. 2 illustrates a distal end of another exemplary EVAC device 200. EVAC device 200 may have similar properties to EVAC device 100, unless stated otherwise herein. Where feasible, 100 has been added to reference numbers related to EVAC device 100 similar to aspects of EVAC device 200. EVAC device 200 may include a multilayered distal body 202 (e.g., a multilayered porous distal body 202) and a vacuum tube 204. Similar to vacuum tube 104, vacuum tube 204 may include an outer wall 208, a lumen 210, and a proximal end of vacuum tube 204 may be connected to a vacuum source (not shown). Outer wall 208 may include a plurality of holes around a circumference of the distal end of vacuum tube 204 and in fluid communication with lumen 210, which may increase the flow of fluid or material from one or more layers of distal body 202 into lumen 210. A distal end 203 of vacuum tube 204 may be attached to distal body 202 via sutures, an adhesive, or the like (including any of the examples discussed above with respect to EVAC device 100). In one example, a recess (not shown) may be provided in distal body 202 to receive distal end 203 of vacuum tube 204.

In some examples, distal body 202 may include an inner core 220 covered or layered with one or more intermediate layer(s) 222 and/or an outer layer 224 (e.g., a mesh outer layer). In FIG. 2, portions of intermediate layer(s) 222 and outer layer 224 are shown as being resected so as to show features of core 220 and/or intermediate layer(s) 222. Core 220 may be referred to herein as one of the layers of distal body 202. Core 220 may be inhibited from contacting tissue at a target site by intervening intermediate layer(s) 222 and/or outer layer 224, while outer layer 224 may be configured to contact the tissue wall at the target site. Core 220 may include an open-cell foam structure with openings 226, having any of the properties of openings 106, discussed above. Openings 226 may be any hole, pore, or channel which provides access to interconnecting channels and/or pores of core 220 to allow for fluid and/or material to flow into lumen 210 of vacuum tube 204. The size of openings 226 may be similar to the size of openings 106. For example, the size of openings 226 may be approximately 500 μm to approximately 1.0 mm in diameter. In some examples, openings 226 may be of a uniform size. Alternatively, openings 226 may vary in size.

Layers of distal body 202 may have various features in order to aid in aspects of an EVAC procedure. The features disclosed below are exemplary and may be used in any combination. In some examples, a single layer 224, 222, 220 may include materials having a plurality of the features described below. Features described as being utilized for one of layers 224, 222, 220 may additionally or alternatively be used for others of the layers.

In some examples, absorbency of distal body 202 may increase from outer layer 224 to core 220. For example, core 220 may be more absorbent than layers 222, 224, such that core 220 may pull fluid or material away from layers 222, 224. In some examples, intermediate layer(s) 222 may be more absorbent than outer layer 224 to pull fluid or material from outer layer 224. Absorbency of distal body 202 may be affected by a density of a material of core 220 and/or layers 222, 224, a size of pores of core 220 and/or layers 222, 224, a density of pores of core 220 and/or layers 222, 224, and/or flexibility of a material of core 220 and/or layers 222, 224.

In aspects, one or more of core 220 or layers 222, 224 may include materials (e.g., ionic materials) that provide storage or collection of exudates. In some examples, such materials may be disposed in two or more of core 220 and/or layers 222, 224, and concentrations of the materials may increase moving inward in a direction toward core 220. A relatively more inner layer may have a greater concentration of such material(s) than a relatively more outer layer. In an example, core 220 may have a higher concentration than layers 222, 224, allowing for core 220 to pull exudate from layers 222, 224. Similarly, intermediate layer(s) 222 may have a higher concentration than outer layer 224 to pull exudate from outer layer 224 into intermediate layer(s) 222. For example, core 220 or layers 222, 224 may include a material comprising alginate, e.g., calcium alginate, sodium carboxy, or methylcellulose. In the presence of fluid or material at the target site, such materials may form a gel or otherwise promote storage or collection of fluid or material within core 220 or layers 222, 224. For example, calcium alginate forms a gel in the presence of fluid, which can retain 20 times its weight. In some examples, alginate may be in a suitable form, e.g., beads, blends, dressings, electrospun scaffolds, flexible fibers, films, foams, gels, hydrogels, microparticles, microspheres, nanoparticles, polyelectrolyte complex, powders, ropes, sheets, and/or sponges, or an injectable form.

In some examples, storage or collection materials, such as those listed above, may be coated onto outer surfaces of one or more of core 220 and/or layers 222, 224 or may be impregnated into core 220 and/or layers 222, 224. Use of such materials may assist with removal of distal body 202. They may assist in stopping bleeding of a tissue (and encouraging wound healing) and preventing or inhibiting coagulated exudate from sticking to another layer of distal body 202. For example, alginate incorporated into core 220 and/or layers 222, 224 may exchange ions with fluids at a treatment site when distal body 202 is disposed at the treatment site. For example, a calcium alginate non-woven core 220 and/or layers 222, 224 may exchange sodium ions in exuding or infected treatment sites (e.g., wounds). Such a core 220 and/or layers 222, 224 may not adhere to a wound bed and may self-adhere to healthy tissue surrounding the wound bed. Furthermore, newly formed tissue may not be affected by washing away the alginate fibers at the wound site.

In an example, intermediate layer(s) 222 may include one or more of the materials listed above or another similar material, which may inhibit intermediate layer(s) 222 from sticking to core 220 and/or outer layer 224.

In some examples, outer surfaces of core 220 and/or layers 222, 224 may be lined with a non-stick material to assist with removal of distal body 202 from a subject's body. Suitable materials include expanded PTFE, perforated PTFE, polyethylene, ChronoFlex C®, and/or PVDF. For example, an outer layer of outer layer 224 may include such a non-stick material.

One or more of core 220 and/or layers 222, 224 may have hydrophobic or hydrophilic properties. In some examples, core 220 may comprise a hydrophobic/non-wetting material, such that fluid or material may flow through openings 226 without moistening inner portions of core 220. A non-wetting core 220 may expedite drainage through lumen 210. In some examples, outer layer 224 may be hydrophilic, to encourage (e.g., wick) fluids to pass into distal body 202. Intermediate layer(s) 222 may also be hydrophobic or hydrophilic in order to encourage flow of fluids. Hydrophilic materials may allow fluids to pass through tighter cellular structures of outer layer 224 and/or intermediate layer(s) 222.

One or more of core 220 or layers 222, 224 (e.g., intermediate layer(s) 222) may include an antibacterial or antimicrobial material, such as silver particles or zinc oxide (ZnO), which may help to limit and/or reduce bacterial growth at a target site. Such antibacterial and/or antimicrobial material(s)/agent(s) may be disposed on a surface of core 220 or layers 222, 224 or may be impregnated into core 220 or layers 222, 224. One or more layers that contact tissue (e.g., outer layer 224 or portions of core 220 or intermediate layer(s) 222 that are not covered by another layer) may include collagen or any other suitable substance to promote tissue growth and structure and thereby encourage healing of a wound.

In some examples, outer layer 224 and/or intermediate layer(s) 222 may have a tight cellular porosity in order to prevent ingrowth through outer layer 224 and/or intermediate layer(s) 222 to a more porous layer thereunder (e.g., core 220 or porous intermediate layer(s) 222). For example, outer layer 224 and/or intermediate layer(s) 222 may be formed of a fine mesh or a cloth. In examples, outer layer 224 and/or intermediate layer(s) 222 may comprise woven or open-cell polymers such as nylon, polyester, urethanes or cellulose, such as natural cotton fibers with sodium sulfate. In aspects, outer layer 224 and/or intermediate layer(s) 222 may be akin to a sock that is positioned around a particular region of core 220. In some examples, the region around which such a sock is positioned may be an entirety of a tissue contact region of distal body 202. In other aspects, outer layer 224 and/or intermediate layer(s) 222 may entirely cover core 220. As discussed above, in some examples, one or more of outer layer 224 and/or intermediate layer(s) 222 may be hydrophilic, such that fluid or material from a target site may flow through the tight cellular porosity of the layer(s), while preventing, e.g., tissue growth into distal body 202. Outer layer 224 also may include protrusions similar to protrusions 114, discussed above with respect to EVAC device 100.

In some examples, portions of an outer surface of outer layer 224 may include securing/anti-migration regions to prevent movement of distal body 202 from the target site. The securing regions may include adhesives, may be textured (e.g., grabbing structures such as micropatterns), and/or may include any structure configured to adhere to a tissue wall. In some examples, the securing regions may be located on the outer surface of outer layer 224, such that the securing regions contact tissue, e.g., only healthy tissue adjacent a target site, e.g., a wound. In some examples, the securing regions may be disposed at a proximal portion of the outer surface of outer layer 224 so as to engage with healthy tissue and avoid interference with disease treatment.

In one example, core 220 may include a large, open cell foam that is non-wetting. Intermediate layer(s) 222 may include an antimicrobial coating or may be impregnated with antimicrobial materials. In alternatives, an outer surface of core 220 may have an antimicrobial coating. Outer layer 224 may include a material with a tight cellular porosity (e.g., a fine mesh) and may include a hydrophilic coating. Outer layer 224 and intermediate layer(s) 222 may have different cellular size and different dispersions. A size of openings or pores in outer layer 224 and/or intermediate layer(s) 222 may be smaller, on average, than a size of openings of core 220. In other words, core 220 may have a larger cellular size than intermediate layer(s) and/or outer layer 224. Dispersion properties of intermediate layer(s) 222 and outer layer 224 may be chosen so to arrange for fluid to move directionally through distal body 202. Although intermediate layer(s) 222 and outer layer 224 are described as being separate layers, it will be appreciated that they may be fixed to one another or otherwise form a single layer.

Outer layer 224 and/or intermediate layer(s) 222 may be secured over core 220 and, in some examples, a portion of distal end 203 of vacuum tube 204. In some examples, a proximal end of outer layer 224 and/or intermediate layer(s) 222 may include an opening 216, such that a distal end of core 220 may be inserted into opening 216 of outer layer 224 and/or intermediate layer(s) 222. Outer layer 224 and/or intermediate layer(s) 222 may then be pulled proximally over core 220 (or core 220 may be moved distally into outer layer 224) to partially or completely cover core 220 and, optionally, a portion of distal end 203 of vacuum tube 204. Outer layer 224 and/or intermediate layer(s) 222 may then be secured to distal end 203 of the vacuum tube 204 by a securement device 240. Securement device 240 may include a clip, drawstring, elastic, suture heat shrinkable capture, or the like.

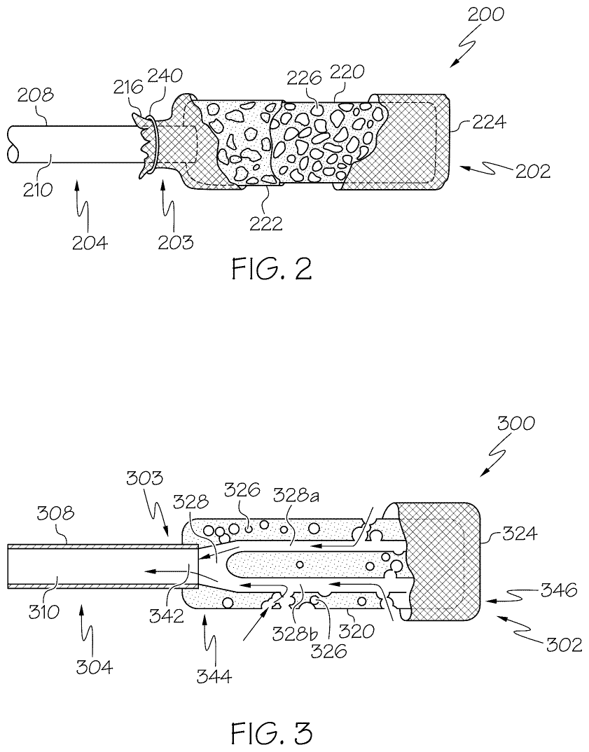

FIG. 3 illustrates a distal end of another exemplary EVAC device 300. FIG. 3 shows a cross-sectional view of EVAC device 300. EVAC device 300 may have any of the properties of EVAC devices 100, 200, unless stated below. Where feasible, like reference numbers add 100 to the reference numbers associated with EVAC device 200. EVAC device 300 may include a multilayered distal body 302 coupled to a distal end 303 of vacuum tube 304. Similar to vacuum tube 104, vacuum tube 304 may include an outer wall 308, a lumen 310, and a proximal end of vacuum tube 304 may be connected to a vacuum source (not shown). A distal end 303 of vacuum tube 304 may be attached to distal body 302 via sutures, an adhesive, or the like (including by any of the mechanisms described above, with respect to EVAC devices 100, 200), such that distal end 303 of vacuum tube 304 is housed within an opening 342 of a main channel 328 formed in a proximal end 344 of distal body 302 or lumen 310 is otherwise in fluid communication with channel 328. Details of channel 328 are discussed below.

In some examples, distal body 302 may include a core 320 and an outer layer 324. Outer layer 324 is shown as being partially resected in FIG. 3 to depict features of core 320. Core 320 may have any of the properties of cores 120, 220, discussed above. A material of core 320 may include polyurethanes (esters and/or ethers), polyvinyl alcohol, composite materials, and/or other medical-grade materials. One or more lumens/channels may be formed within core 320 and in fluid communication with lumen 310 of vacuum tube 304. As shown in FIG. 3, core 320 may include channels 328, 328a and 328b. As explained above, channel 328 may be at proximal end 344 of core 320 and may receive distal end 303 of vacuum tube 304 or otherwise be fluidly coupled to lumen 310. Channels 328a, 328b may be branches/forks of main channel 328. Channels 328a and 328b may extend proximally from distal end 346 of core 320 to proximal end 344 of core 320. A distal end of channels 328a, 328b may terminate proximally of a distalmost end of core 320, such that channels 328a, 328b have closed distal ends. Alternatively, channels 328a, 328b may extend fully through distal end 346 of core 320, such that channels 328a, 328b have open distal ends. Channels 328a and 328b may converge at proximal end 344 of core 320 forming main channel 328 adjacent to lumen 310. Main channel 328 may extend through proximal end 344 of core 320, such that main channel 328 has an open proximalmost end. It will be appreciated that distal body 302 may include any number of channels converging into a main channel at proximal end 344 of distal body 302. The channels may have any suitable arrangement (e.g., may be symmetrical, asymmetrical, etc.). Openings 326 may be dispersed on an outer surface of core 320 and within core 320. Openings 326 may be of varying sizes and shapes, and may be in fluid communication with channels 328, 328a, 328b. Channels 328, 328a, 328b may be sized and shaped so that they do not collapse upon application of negative pressure from vacuum tube 304. For example, channels 328, 328a, 328b may have a relatively small diameter/width so as to prevent their collapse. A size of channels 328, 328a, 328b may be sufficiently large/wide, such that fluids may pass therethrough. Thus, when negative pressure is supplied to vacuum tube 304, fluid or material may flow from a target site, through openings 326, through channels 328, 328a, 328b, and into lumen 310 of vacuum tube 304. Channels 328, 328a, 328b may provide for increased flow to lumen 310, as compared with a core such as core 220.

Outer layer 324 may have any of the properties of layers 222, 224, discussed above. Similarly, layers 222, 224 may have any of the features of outer layer 324, discussed below. In some examples, outer layer 324 may be formed of a fine mesh or a cloth. Similar to outer layer 224, a material of outer layer 324 may comprise woven or open-cell polymers such as nylon, polyester, urethanes or cellulose, such as natural cotton fibers with sodium sulfate. Outer layer 324 may completely surround or cover core 320, similar to outer layer 224. In other examples, outer layer 324 may only partially cover core 320.

In some examples, outer layer 324 may be formed of sections with varying materials, e.g., nylon, polyester, urethanes, and/or cellulose. In some examples, size openings and dispersion of openings on each section of outer layer 324 may vary from section to section. In some examples, sections of outer layer 324 may be independent or in communication with one another. In aspects, outer layer 324 may include a plurality of layers of material, which may be formed of the same material or from different materials having different cellular size and/or different dispersion features. Similar to outer layer 224, outer layer 324 may be hydrophilic, such that fluid or material from a target site may flow through the small openings of outer layer 324, while preventing, e.g., tissue growth into distal body 302.

Outer layer 324 may cooperate with core 320 to move fluids directionally through distal body 302. For example, fluids may flow along a path of least resistance, through channels 328, 328a, 328b. Fluids may enter channels 328, 328a, 328b through sides (e.g., radially outer sides) of channels 328, 328a, 328b, as shown by the arrows in FIG. 3, via openings 326. Fluids may enter openings 326 through pores/openings/perforations of outer layer 324. Fluids also may enter through distal ends of channels 328a, 328b, through openings 326 on distal end 346 of core 320 (via, e.g., openings on a distal side of outer layer 324).

Cores 220, 320 and layers 222, 224, 324 of distal bodies 202, 302 may have varying hydrophilic and hydrophobic properties. For example, a layer that is preferably hydrophilic may allow for fluid or material to flow through small openings formed on a layer or pull fluids or material from an outer layer. This may increase lubricity and prevent or inhibit clogging of distal bodies 202, 302 by allowing exudate to pass through the porous structures of distal bodies 202, 302. In some examples, a layer that is preferably hydrophobic may allow for fluid or material to flow through openings of a layer without moistening inner portions of the layer. In some examples, cores 220, 320 may have a larger open cell/pore structure and/or a more loose open cell structure than layers 222, 224, 324 to allow more fluid or material to flow through openings 226, 326 and into lumens 210, 310 when negative pressure is supplied to lumens 210, 310. A larger open cell structure and/or a loose open cell structure of cores 220, 320 may reduce a pressure gradient through a thickness of distal bodies 202, 302 and/or may allow for a greater collapse of distal bodies 202, 302 during delivery to a target site. In some examples, layers 222, 224, 324 may be more rigid than cores 220, 320 and/or have smaller pore sizes than cores 220, 320 to optimize granulation tissue formation and/or control tissue ingrowth. Alternatively, layers 222, 224, 324 may be flexible to allow distal bodies 202, 302 to fit into smaller (e.g., more microscopic), irregular spaces. It will be appreciated that distal bodies 202, 302 may include any combination of cores 220, 320 and layers 222, 224, 324 described above to reduce the number of distal body 202, 302 exchanges during an EVAC procedure and otherwise improve EVAC procedures.

It will be apparent to those skilled in the art that various modifications and variations may be made in the disclosed devices without departing from the scope of the disclosure. Other aspects of the disclosure will be apparent to those skilled in the art from consideration of the specification and practice of the features disclosed herein. It is intended that the specification and examples be considered as exemplary only.

We claim:

1. A medical device comprising:
a distal body coupled to a distal end of a vacuum tube, wherein the distal body is in fluid communication with a lumen of the vacuum tube, the distal body comprising:
a core including a plurality of openings; and
one or more layers surrounding the core.

2. The medical device of claim 1, wherein at least one of the one or more layers defines a plurality of openings.

3. The medical device of claim 2, wherein the plurality of openings of the one or more layers has a smaller average size than the plurality of openings of the core.

4. The medical device of claim 1, wherein the one or more layers completely surround the core.

5. The medical device of claim 1, wherein the one or more layers surround only a portion of the core.

6. The medical device of claim 1, wherein the one or more layers are coated with an antimicrobial material.

7. The medical device of claim 1, wherein at least one of the one or more layers is coupled to the distal end of the vacuum tube via a clip, drawstring, or suture.

8. The medical device of claim 1, wherein at least one of the one more layers includes one or more protrusions extending radially outward from an outer surface of the at least one of the one or more layers.

9. The medical device of claim 1, wherein the one or more layers include a first layer surrounding the core and a second layer surrounding the first layer.

10. The medical device of claim 9, wherein an absorbency of the second layer is greater than an absorbency of the first layer, and wherein the absorbency of the first layer is greater than an absorbency of the core.

11. The medical device of claim 9, wherein the core includes a hydrophobic material, and wherein the first layer and the second layer include a hydrophilic material.

12. The medical device of claim 9, wherein a proximal portion of an outer surface of the second layer includes an adhesive.

13. The medical device of claim 1, wherein the one or more layers are coated with a material comprising alginate.

14. The medical device of claim 1, wherein the core includes a first channel and a second channel, wherein the first channel and the second channel extend from a proximal end of the core to a distal end of the core, and wherein a distal end of each of the first channel and the second channel terminate proximal of a distalmost end of the core.

15. The medical device of claim 14, wherein the first channel and the second channel converge at the proximal end of the core to form a third channel.

16. A medical device comprising:
a distal body coupled to a distal end of a vacuum tube, wherein the distal body is in fluid communication with a lumen of the vacuum tube, the distal body comprising:
a core including:
a plurality of openings disposed on an outer surface of the core;
a first channel; and
a second channel, wherein the first channel and the second channel converge at a proximal end of the core to form a third channel; and
at least one layer surrounding the core.

17. The medical device of claim 16, wherein a distal end of each of the first channel and the second channel is closed.

18. The medical device of claim 16, wherein the third channel is adjacent to the lumen of the vacuum tube.

19. A medical device comprising:
a distal body coupled to a distal end of a vacuum tube, the distal body comprising:
a core including a plurality of openings in fluid communication with a lumen of the vacuum tube;
a first layer surrounding the core; and
a second layer surrounding the first layer, wherein each of the first layer and the second layer include a plurality of openings, and wherein the plurality of openings of each of the first layer and the second layer have a smaller average size than the plurality of openings of the core.

20. The medical device of claim 19, wherein an outer surface of the second layer includes a plurality of protrusions extending radially outward.
